(19) United States
(12) Patent Application Publication
(10) Pub. No.: US 2026/0053734 A1
(43) Pub. Date: Feb. 26, 2026

(54) METHODS OF FABRICATING THERMOSENSITIVE HYDROGELS AND USES THEREOF FOR INDUCING CHRONIC GLAUCOMA ANIMAL MODELS

(71) Applicant: The Second Xiangya Hospital of Central South University, Changsha (CN)

(72) Inventors: Ke LIU, Changsha (CN); Rong LIU, Changsha (CN); Feng ZHANG, Changsha (CN); Xuanchu DUAN, Changsha (CN); Yiwen WANG, Changsha (CN)

(73) Assignee: The Second Xiangya Hospital of Central South University, Changsha (CN)

(21) Appl. No.: 19/309,512

(22) Filed: Aug. 25, 2025

(30) Foreign Application Priority Data
Aug. 23, 2024 (CN) 202411169012.7

(51) Int. Cl. A61K 9/00; A61K 38/21; A61K 47/36

(52) U.S. Cl. CPC A61K 9/0024; A61K 9/0048; A61K 38/215; A61K 38/217; A61K 47/36

(57) ABSTRACT

The present disclosure provides a preparation method of a thermosensitive hydrogel and application thereof in establishing a chronic glaucoma model, falling within the technical field of medicine preparation and disease model development. In the present disclosure, an animal model characterized by straightforward procedures, a short induction period, sustained and stable intraocular pressure elevation, and high modeling success rates is established, which closely recapitulates the pathophysiology of chronic open-angle glaucoma, addresses limitations associated with repeated short-term modeling, including ocular tissue damage and inflammatory responses, and demonstrates high potential for translational applications.

### CROSS-REFERENCE TO RELATED APPLICATIONS

The application claims priority to Chinese patent application No. 202411169012.7, filed on Aug. 23, 2024, the entire contents of which are incorporated herein by reference.

### TECHNICAL FIELD

The present disclosure relates to the technical field of medical material fabrication and disease model development, specifically disclosing methods of fabricating thermosensitive hydrogels and uses thereof for inducing novel chronic glaucoma animal models.

### BACKGROUND

Glaucoma, the second leading cause of global blindness, is an irreversible neurodegenerative optic neuropathy characterized by progressive retinal ganglion cell (RGC) death and visual field defects. Primary open-angle glaucoma (POAG) accounts for 60-70% of all glaucoma cases, though its complex pathogenesis remains incompletely elucidated, complicating early diagnosis. Pathological elevation of intraocular pressure (IOP) remains the most significant risk factor for glaucomatous neurodegeneration, and IOP reduction is the only controllable therapeutic strategy. In glaucoma research, animal models recapitulating disease-specific features are indispensable tools for deciphering injury mechanisms and evaluating interventions. Recent advances in induction methodologies and quantitative damage assessment have yielded multiple etiologically diverse animal models that mirror key pathophysiological hallmarks of human glaucoma.

Existing animal models of ocular hypertension-induced glaucoma include, but are not limited to, the following:

1. Anterior/Posterior Chamber Injection Models: Injections of specific obstructive materials (e.g, microbeads, silicone oil, etc.) acutely elevate IOP by trabecular meshwork occlusion. Limitations include transient and unstable IOP elevation, requirement for repeated injections, and frequent complications (e.g, anterior chamber inflammation, corneal endothelial decompensation, lens/iris injury, etc.).

2. Laser Photocoagulation Models: Laser ablation of trabecular meshwork(TM) or episcleral veins induces IOP elevation but requires specialized equipment, demonstrates short-term efficacy, and exacerbates intraocular inflammation with repeated laser treatments.

3. Intravitreal Pharmacological Models: Cytokine injections (e.g, TGF-β, corticosteroids, etc.) risk retinal toxicity and endophthalmitis due to frequent invasive procedures.

4. Transgenic Models: DBA/2J (GpnmbR150X/Tyrp1isa mutations) or Tg-MYOCY437 mice exhibit chronic IOP elevation but require prolonged breeding cycles and high maintenance costs.

None fully recapitulate the chronic, progressive nature of human primary open-angle glaucoma (POAG). Thus, developing a physiologically relevant animal model that mirrors human POAG pathogenesis is critical for elucidating disease mechanisms and advancing therapeutic discovery.

Interferons (IFNs) are bioactive polypeptides that modulate cellular functions, exhibiting antiviral and immunoregulatory properties. They primarily participate in cell proliferation, immune responses, and hematopoiesis. Based on cellular origin and biological activity, IFNs are classified into three types: Type I (mainly IFN-α/β, with rapid antiviral effects), Type II (IFN-γ, also termed immune interferon, possessing potent antitumor and immunomodulatory activities), and Type III (IFN-k, inducing localized antiviral immunity).

Studies report that DDX58 mutations induce TM cytotoxicity and disrupt cytoskeletal protein expression, accompanied by elevated Type I IFN production. Notably, IFN-γ levels are significantly elevated in the aqueous humor of POAG patients. Intracameral injection of Zika virus in mice triggers substantial IFN expression and marked IOP elevation. Based on our research team's preliminary exploration of mechanisms, we found that IFN-γ suppresses TM cell viability via necroptosis activation, while IFN-β₁ promotes autophagic TM cell death. Intracameral injection of either of these cytokines induce IOP elevation, which in turn leads to glaucomatous neurodegeneration.

However, single injections of free IFN-γ or IFN-β₁ fail to sustain stable IOP elevation due to rapid aqueous humor turnover and drug clearance, necessitating weekly (or more frequent) reinjections to maintain effective concentrations. Repeated procedures risk anterior segment trauma and exacerbated inflammation. To address this, we developed a thermosensitive hydrogel loaded with IFN-γ or IFN-β₁ for sustained intracameral delivery. This system enables prolonged cytokine release, minimizes iatrogenic injury, and establishes a novel chronic OAG animal model.

### SUMMARY

The present disclosure aims to provide methods of fabricating thermosensitive hydrogels and uses thereof for inducing novel chronic glaucoma animal models. This approach offers significant advantages, including a simplified operational procedure, shortened induction period, sustained and stable IOP elevation, high modeling success rate, and close recapitulation of the pathophysiological features of chronic OAG. It addresses limitations associated with repeated short-term modeling interventions, thereby demonstrating substantial translational potential.

The above technical objective of the present disclosure is achieved by the following technical solution, a fabrication method for thermosensitive hydrogels, including the following steps:
S1, synthesis, dialysis, and lyophilization of methacrylated hyaluronic acid (HA-MA);
S2, synthesis, dialysis, and lyophilization of thermosensitive hydrogel;
S3, gelation characterization and injectability assessment;
S4, scanning electron microscopy (SEM) analysis of hydrogel microstructure;
S5, fourier transform infrared (FT-IR) spectroscopic analysis of hydrogel;
S6, determination of hydrogel swelling ratio and thermosensitivity evaluation;
S7, hydrogel degradation rate assessment; and
S8, synthesis of IFN-loaded sustained-release thermosensitive hydrogel and release kinetics profiling.

Step S1 includes the following specific steps:
1, synthesis of HA-MA, dissolve 2 g of hyaluronic acid (HA) in 200 mL deionized water, then promptly add 15 mL methacrylic anhydride (MA). Adjust the pH to 8-12 using 5M NaOH, and react at 2-6° C. for 12 hours;
2, the synthesized HA-MA conjugate was dialyzed using cellulose ester dialysis membranes for 4 days, with the dialysate replaced three times daily;
3, the modified HA-MA conjugate was pre-frozen at −80° C. and subsequently lyophilized using a freeze-dryer.

Step S2 includes the following specific steps:
1, 0.2 g of lyophilized HA-MA from Step S1 was weighed and homogenized with 180 mL deionized water in a 250 mL round-bottom flask until complete dissolution was achieved;
2, 7 mL of 10% (w/v) ethyl methacrylate (EMA) and 7 mL of 10% (w/v) potassium persulfate (KPS) were added to round-bottom flask as the initiator system. Subsequently, 7 mL of 4% (v/v) N,N,N′,N′-tetramethylethylenediamine (TEMED), pre-deoxygenated with nitrogen, was introduced as the cocatalyst under continuous positive nitrogen pressure. Copolymerization proceeded at 20° C. for 24 hours to form the copolymer network;
3, the synthesized sample was dialyzed using cellulose ester dialysis membranes for 4 days, with the dialysate replaced three times daily;
4, the synthesized thermosensitive conjugate was pre-frozen at −80° C. and subsequently lyophilized using a freeze-dryer.

Step S3 includes the following specific steps:
1, the sol-gel transition was determined using the inverted vial method. The prepared hydrogel solution was heated from 30° C. at a rate of 1° C./min. Every 30 seconds, the vial was tilted to a 600 angle relative to the horizontal plane. Gelation was defined as the point at which no flow was observed for 30 seconds. The gelation temperature (phase transition temperature) was identified to be 37° C. Subsequently, the sample was equilibrated in a water bath set to 37° C. and tilted every 5 seconds at the same angle to confirm the gelation temperature and record the precise gelation time; and
2, the modified solid sample from step S3.1 was dissolved in deionized water to prepare HA hydrogels at various concentrations. The resulting solutions were evaluated for their gelation capability at 37° C. and injectability based on retained flowability under appropriate shear conditions.

Step S4 includes the following specific steps:
the solid sample from step S1 and S2 was sectioned by either scissor-cutting or cryo-fracture in liquid nitrogen to obtain cross-sectional specimens. The samples were affixed to the specimen stage using conductive adhesive. After purging and evacuating the chamber to achieve high vacuum, the acceleration voltage was increased while simultaneously raising the stage to an appropriate working distance. Sample location was identified, and imaging was performed using field-emission scanning electron microscopy (FE-SEM). Micrographs were acquired at various magnifications with scale bars for subsequent microstructural analysis.

Step S5 includes the following specific steps:
1, hydrogel samples were prepared according to step S1 and S2. The samples were thoroughly dried and trimmed to a diameter greater than 0.5 cm. The FT-IR spectrometer and its accompanying software were initialized, and background scans were performed under standardized parameters to establish a baseline. Spectra of the samples were then acquired and recorded; and
2, the resulting spectral data were analyzed to identify the characteristic functional groups present in the material. Further analysis of the chemical composition of thermosensitive HA hydrogel.

Step S6 includes the following specific steps:
1, the equilibrium swelling ratio of the hydrogels was determined gravimetrically. Lyophilized thermosensitive gel samples, prepared according to Steps S1 and S2, were first weighed (denoted as Wd). They were then immersed in phosphate-buffered saline (1×PBS, pH=7.4) heated at temperatures ranging from 20° C. to 45° C. After swelling for 24 hours at a predetermined temperature, the samples were removed, promptly blotted with filter paper to remove excess surface water, and weighed again (denoted as Ws);
2, the equilibrium swelling ratio (ESR) of the hydrogel was calculated using the following formula:

$$ESR = ( Ws - Wd ) / Wd \times 100 \%$$

(Ws represents the mass of the hydrogel at equilibrium swelling, and Wd denotes the mass of the dry gel prior to swelling); and
3, upon reaching physiological temperature (37° C.), the hydrogel exhibits reduced and stabilized swelling, thereby maintain the sustained release of its encapsulated cytokine payload.

Step S7 includes the following specific steps:
1, the degradation profile of the hydrogel was assessed gravimetrically. A pre-formed 500 μL thermosensitive HA hydrogel sample was weighed (denoted as Wo) and immersed in 20 mL of PBS (1×PBS, pH 7.4, The system was maintained at 37° C. in a constant-temperature water bath, with the PBS solution replaced daily. At predetermined time intervals, the sample was removed, carefully blotted with filter paper to remove surface moisture, and reweighed (denoted as Wt);
2, the degradation ratio was calculated using the following formula:

$$WR = ( Wo - Wt ) / Wo \times 100 \%$$

(Wo represents the initial mass and Wt denotes the degraded mass); and
3, under physiological temperature (37° C.), the HA hydrogel underwent gradual and sustained degradation over time, exhibiting a relatively constant degradation rate. By day 28, the degradation ratio reached approximately 66.3%. Throughout this process, the encapsulated IFN cytokine was released in a progressive manner.

Step S8 includes the following specific steps:
1, synthesis of IFN-loaded thermosensitive hydrogel for sustained release: Based on previous findings from our group regarding the effects of IFN-β₁ and IFN-γ on TM cells, the targeted loading concentrations were set at 2,500-5,000 U/mL for IFN-β₁ and 20-30,000 U/mL for IFN-γ. Each IFN was incorporated into the prepared thermosensitive HA hydrogel solution. The in vitro release profiles were subsequently evaluated under physiologically relevant conditions (37° C.);
2, in vitro release kinetics were assessed as follows: 1 mL IFN-loaded thermosensitive hydrogel were placed in dialysis bags and immersed in 100 mL of PBS solution. The system was maintained at 37° C. under constant agitation at 100 rpm. At predetermined time intervals, 50 μL aliquots of the release medium were collected and replaced with an equal volume of fresh PBS to maintain sink conditions. All samples were stored at −20° C. until analysis. The concentrations of IFN-γ and IFN-β₁ were quantified using corresponding ELISA kits (abcam); and
3, the release profiles demonstrated that hydrogels loaded with 30,000 U/mL IFN-γ and 5,000 U/mL IFN-β₁ achieved cumulative release rates of 92.0% and 86.7%, respectively, over 28 days. These results confirm the capability of the loaded hydrogel to sustain the release of encapsulated agents for approximately one month or longer, supporting its potential for long-acting release following a single administration.

The present disclosure is further configured as a method for establishing a chronic glaucoma animal model using a thermosensitive hydrogel, characterized by including the following steps:
S1, pre-modeling preparation;
S2, induction of animal models; and
S3, detection of ocular changes induced in the animal model were consistent with the characteristics of chronic open-angle glaucoma.

Step S1 includes the following specific steps:
1, experimental animal preparation: C57BL/6J mice (6-8 weeks old, male) were housed in a specific pathogen-free (SPF) barrier facility with ad libitum access to food and water. After a 7-day acclimation period, experiments were initiated in strict compliance with institutional animal care and use guidelines. At 08:00-10:00 a.m. on the day prior to surgery, intraocular pressure (IOP) was measured in inhalation-anesthetized mice using a TonoLab rebound tonometer. The mean value was recorded as baseline IOP. All subsequent IOP measurements were performed by the same operator within the identical daily time window; and
2, preparation of drug-loaded thermosensitive hydrogel for sustained release: the target loading concentrations were 2,500-5,000 U/mL for IFN-β₁ and 20-30,000 U/mL for IFN-7.

Step S2 includes the following specific steps:
1, experimental animal groups: The model groups included intracameral injection of IFN-7-loaded sustained-release thermosensitive hydrogel and intracameral injection of IFN-β₁-loaded sustained-release thermosensitive hydrogel. The control group received intracameral injection of drug-free thermosensitive hydrogel;
2, anesthesia of experimental animals: anesthesia was induced in both model and control group mice via intraperitoneal injection of 1% pentobarbital sodium (40-50 mg/kg). The periocular area was disinfected with iodine swabs, eyelashes were trimmed, and the eyeball was exposed. Ocular surfaces were irrigated with sterile phosphate buffered saline(PBS) or ofloxacin ophthalmic solution, followed by drying with sterile cotton swabs. Prior to surgery, topical 0.5% proparacaine hydrochloride was administered until abolition of withdrawal reflexes confirmed adequate general and local anesthesia; and
3, intracameral injection in experimental animals: a microsurgical technique was employed for intracameral hydrogel delivery. First, the eyeball was stabilized with micro-forceps, a 150 corneal paracentesis knife was used to create a corneal limbal side incision, and aqueous humor was partially drained by the side incision. Then, a 30G needle attached to a micro syringe was inserted through the side incision at a slight angle, and approximately 3 μL of hydrogel was injected into the anterior chamber. This was followed by slow injection of 1 μL air to ensure complete hydrogel delivery and formation of a small intracameral bubble. The needle was maintained in situ for >30 s before gradual withdrawal, allowing the air bubble to self-seal the corneal incision and prevent reflux. The bubble was spontaneously absorbed within hours. Special care was taken to avoid iris or lens capsule injury during the procedure. Postoperatively, ofloxacin ophthalmic ointment was applied to prevent infection. Mice were placed on a thermoregulated recovery pad until spontaneous awakening before returning to housing. Reinjections were performed based on hydrogel degradation kinetics (monitored via slit-lamp) and IOP fluctuations to maintain therapeutic drug concentrations. The total modeling period was 3 months to establish stable chronic ocular hypertension.

Step S3 includes the following specific steps:
1, intraocular pressure (IOP) measurement: IOP was measured in both model and control group mice using a TonoLab rebound tonometer on days 1, 2, 3, 5, 7, 9, 11, 14, 17, 20, 24, and 28 post-modeling. Provided IOP stabilized, measurements were subsequently taken twice weekly during consistent time windows. Based on the degradation profile of the hydrogel in the anterior chamber and observed IOP fluctuations, additional intracameral injections of various IFN-loaded hydrogels were administered to maintain therapeutic IFN concentrations. All acquired IOP data were subjected to statistical analysis to evaluate temporal changes;
2, distribution of the hydrogel in the anterior chamber was monitored using a ZEISS ophthalmic surgical microscope immediately after modeling, and at 1, 2, 4, 6, 8, 10, and 12 weeks post-modeling in both model and control group mice. Images were captured for subsequent analysis of gelation status, degradation progression, and any signs of anterior segment inflammation;
3, at the end of the first, second, and third months post-modeling, subsets of animals from both the model and control groups were euthanized. The enucleated eyes were processed for paraffin embedding and sectioning. Following hematoxylin and eosin (H&E) staining, the sections were examined and imaged using light microscopy. A comparative histological analysis was conducted to evaluate morphological alterations in the corneal layers, iridocorneal angle configuration, cytoarchitectural organization and cellularity of the retinal layers, depth of the optic disc cupping, and the extent of retrobulbar optic nerve atrophy;
4, retinal ganglion cells (RGCs) quantification: At the end of months 1, 2, and 3 post-modeling, subsets of animals from both model and control groups were euthanized via transcardial perfusion. The enucleated eyes were processed for whole-retina flat-mount preparation and immunostaining for RGCs. The posterior pole, mid-periphery, and peripheral regions across all four retinal quadrants were imaged using a ZEISS microscope. Immunopositive RGCs were counted, and the data were subjected to statistical analysis. RGCs loss in the model group, compared to the control group, was assessed to evaluate the extent of optic nerve injury in this ocular hypertension model;
5, transmission electron microscopy (TEM) of TM structure: At the end of the first, second, and third months post-modeling, subsets of animals from both model and control groups were euthanized. A 2 mm×4 mm tissue segment containing the iridocorneal and TM was dissected from the enucleated eyes. The specimens were fixed in 2.5% glutaraldehyde and processed for TEM to examine the ultrastructural details of the TM. Comparative ultrastructural analysis between the model and control groups suggested that IFN-γ and IFN-β₁ may contribute to the pathogenesis of open-angle glaucoma by potentially inducing TM injury and suppressing cellular activity; and
6, assessment of retrobulbar optic nerve axonal degeneration by immunohistochemistry: At the end of months 1, 2, and 3 post-modeling, subsets of animals from the model and control groups undergo transcardial perfusion. A 1.5-2.0 mm segment of the optic nerve (ON) posterior to the globe was dissected and fixed in situ with 2% glutaraldehyde and 2% paraformaldehyde. The specimens were sectioned and stained with 1% paraphenylenediamine (PPD). Sections were imaged using a 100×objective on a ZEISS fluorescence microscope to tile the entire ON area without overlap. From the center of each image, at least two non-overlapping regions (21.4 μm×29.1 μm) were selected, and the number of intact axons within these areas was manually quantified. The average number of surviving axons per ON was calculated from all images of a single nerve. The mean number of surviving axons in the model group was compared to that of the control group to determine the percentage of axonal survival. Comparative analysis of axonal structural degeneration between the two groups was also performed.

In summary, the present disclosure offers the following advantages: It establishes a disease animal model characterized by simple procedures, a short induction period, sustained and stable elevation of IOP, and a high success rate, which more accurately recapitulates the pathophysiological alterations of chronic OAG. This approach mitigates issues associated with repeated short-term modeling maneuvers, such as ocular tissue damage and inflammatory responses, thereby demonstrating high potential for translational applications.

### BRIEF DESCRIPTION OF THE DRAWINGS

FIG. 1A shows the results of the gel formation experiment;

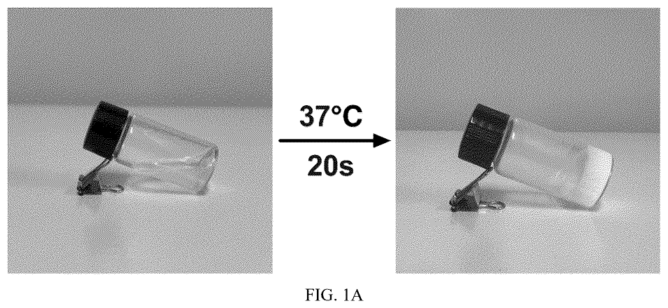

FIG. 1B shows the scanning electron microscopy results;

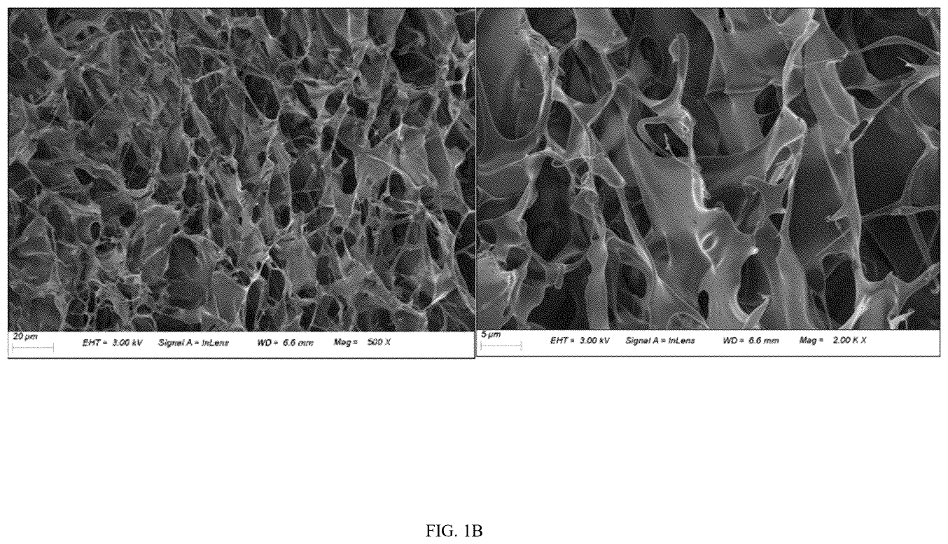

FIG. 1C shows the Fourier transform infrared spectroscopy results;

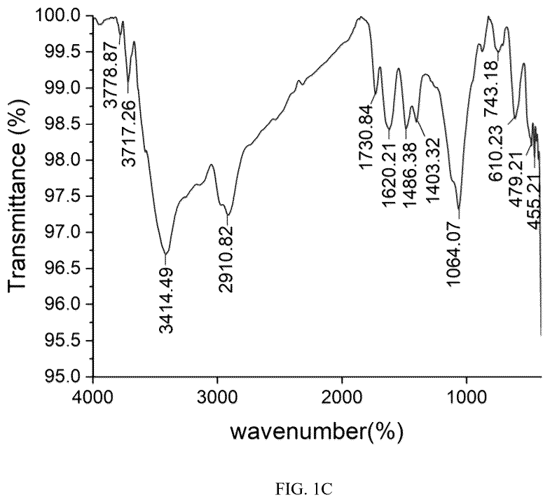

FIG. 1D shows the swelling results at different temperatures;

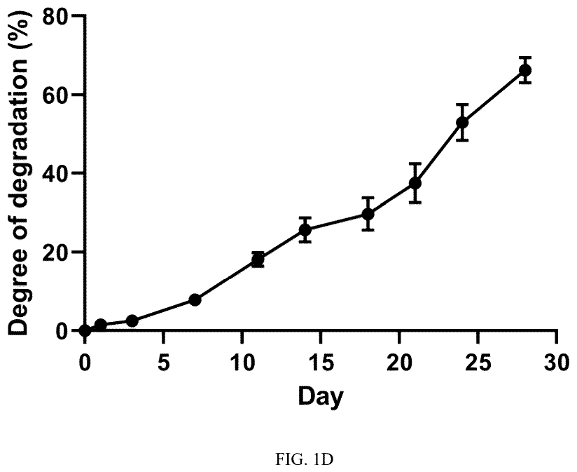

FIG. 1E shows the in vitro degradation rate results;

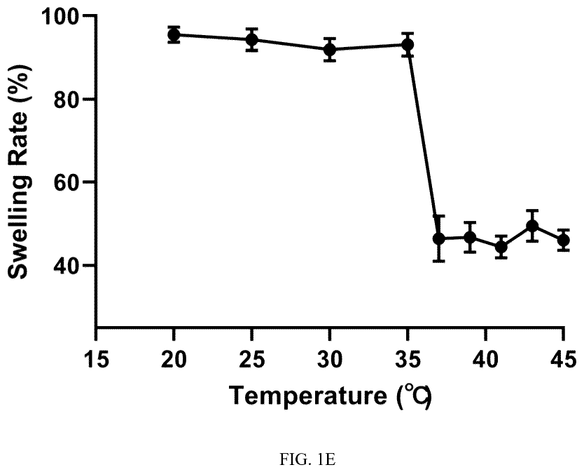

FIG. 1F shows the in vitro release rate results of hydrogel loaded with IFN-γ (30000 U/ml);

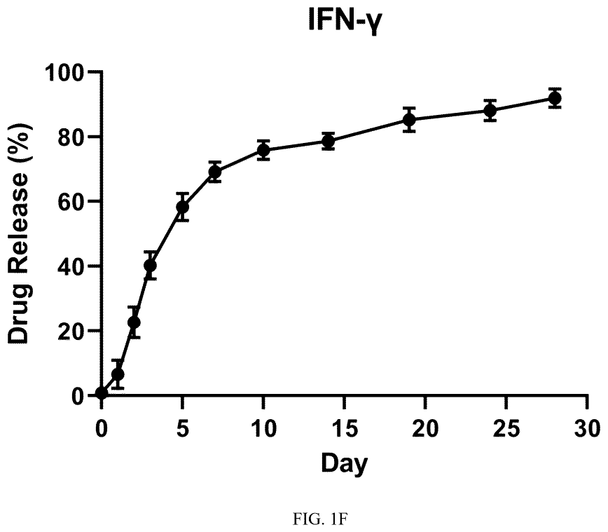

FIG. 1G shows the in vitro release rate results of hydrogel loaded with IFN-β₁ (5000 U/ml);

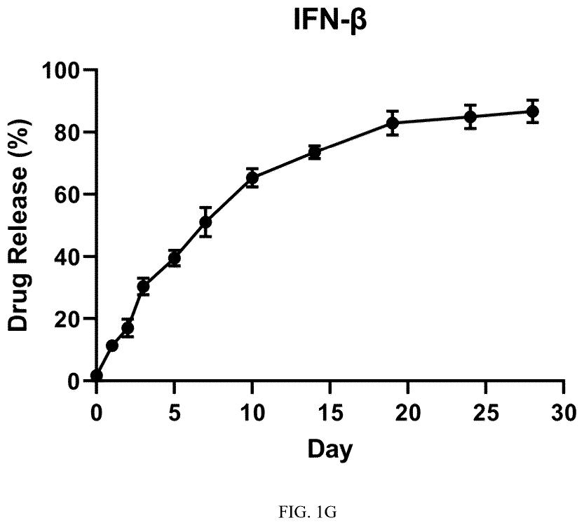

FIG. 2A shows the intraocular pressure measurement results after intracameral injection of hydrogel loaded with IFN-γ (30000 U/ml);

FIG. 2B shows the intraocular pressure measurement results after intracameral injection of hydrogel loaded with IFN-β₁ (5000 U/ml);

FIG. 2C shows the distribution and degradation of the anterior chamber after intracameral injection in mice; and

FIG. 2D shows the HE staining of eyeball sections from model mice to observe tissue changes.

### DETAILED DESCRIPTION OF THE EMBODIMENTS

With reference to the accompanying figures, the present disclosure is described in further detail below.

Example 1, a fabrication method for thermosensitive hydrogels, including the following steps.

S1. Synthesis, dialysis, and lyophilization of HA-MA, includes the following specific steps.
1. Synthesis of HA-MA, dissolve 2 g of HA in 200 mL deionized water, then promptly add 15 mL MA. Adjust the pH to 8-12 using 5M NaOH, and react at 2-6° C. for 12 hours.
2. The synthesized HA-MA conjugate was dialyzed using cellulose ester dialysis membranes for 4 days, with the dialysate replaced three times daily.
3. The modified HA-MA conjugate was pre-frozen at −80° C. and subsequently lyophilized using a freeze-dryer.

S2. Synthesis, dialysis, and lyophilization of thermosensitive hydrogel, includes the following specific steps.
1. 0.2 g of lyophilized HA-MA from Step S1 was weighed and homogenized with 180 mL deionized water in a 250 mL round-bottom flask until complete dissolution was achieved.
2. 7 mL of 10% (w/v) EMA and 7 mL of 10% (w/v) KPS were added to round-bottom flask as the initiator system. Subsequently, 7 mL of 4% (v/v) TEMED, pre-deoxygenated with nitrogen, was introduced as the cocatalyst under continuous positive nitrogen pressure. Copolymerization proceeded at 20° C. for 24 hours to form the copolymer network.
3. The synthesized sample was dialyzed using cellulose ester dialysis membranes for 4 days, with the dialysate replaced three times daily.
4. The synthesized thermosensitive conjugate was pre-frozen at −80° C. and subsequently lyophilized using a freeze-dryer.

S3. Gelation characterization and injectability assessment, includes the following specific steps.
1. The sol-gel transition was determined using the inverted vial method. The prepared hydrogel solution was heated from 30° C. at a rate of 1° C./min. Every 30 seconds, the vial was tilted to a 60° angle relative to the horizontal plane. Gelation was defined as the point at which no flow was observed for 30 seconds. The gelation temperature (phase transition temperature) was identified to be 37° C. Subsequently, the sample was equilibrated in a water bath set to 37° C. and tilted every 5 seconds at the same angle to confirm the gelation temperature and record the precise gelation time. Results indicated that the hydrogel formed a gel within 20 seconds under in vitro conditions at 37° C.
2. The modified solid sample from step S3.1 was dissolved in deionized water to prepare HA hydrogels at various concentrations (e.g, 1%, 3%, 5%; final solid content was adjusted based on gelation behavior and microscopic morphology). The resulting solutions were evaluated for their gelation capability at 37° C. and injectability based on retained flowability under appropriate shear conditions. Results demonstrated that the 1% HA formulation failed to gel, while the 5% HA formulation gelled but exhibited insufficient injectability due to poor flow. Consequently, the 3% HA formulation was identified as the optimal concentration, demonstrating effective gelation and suitable injectability.

S4. SEM analysis of hydrogel microstructure, includes the following specific steps:
1. The solid sample from step S1 and S2 was sectioned by either scissor-cutting or cryo-fracture in liquid nitrogen to obtain cross-sectional specimens. The samples were affixed to the specimen stage using conductive adhesive. After purging and evacuating the chamber to achieve high vacuum, the acceleration voltage was increased while simultaneously raising the stage to an appropriate working distance. Sample location was identified, and imaging was performed using FE-SEM. Micrographs were acquired at various magnifications with scale bars for subsequent microstructural analysis.
2. Results indicated that the hydrogel exhibited a porous, network-like architecture, consistent with the typical structural characteristics of hydrogels. This configuration permits cytokines or drugs to diffuse through the matrix, thereby facilitating cytokines or drugs release.

S5. FT-IR spectroscopic analysis of hydrogel, includes the following specific steps.
1. Hydrogel samples were prepared according to step S1 and S2. The samples were thoroughly dried and trimmed to a diameter greater than 0.5 cm. The FT-IR spectrometer and its accompanying software were initialized, and background scans were performed under standardized parameters to establish a baseline. Spectra of the samples were then acquired and recorded.
2. The resulting spectral data were analyzed to identify the characteristic functional groups present in the material. Further analysis of the chemical composition of thermosensitive HA hydrogel.
3. The thermosensitive hydrogel was synthesized via the synthesis reaction was initiated by KPS with TEMED as a catalyst, leading to the formation of a thermosensitive hydrogel via free radical polymerization mediated by 504. FT-IR analysis of the hydrogel material revealed characteristic absorption bands at 3414 cm⁻¹ (O—H stretch), 2910 cm⁻¹ (C—H stretch), and a strong band at 1064 cm⁻¹ (C—O—C stretch). Minor absorption bands were observed at 1730 cm⁻¹ (C═O stretch) and 1620 cm⁻¹ (N—H bend). These spectral features confirm the chemical functional group composition of the hydrogel.

S6. Determination of hydrogel swelling ratio and thermosensitivity evaluation, includes the following specific steps.
1. The equilibrium swelling ratio of the hydrogels was determined gravimetrically. Lyophilized thermosensitive gel samples, prepared according to Steps S1 and S2, were first weighed (denoted as Wd). They were then immersed in PBS (1×PBS, pH=7.4) heated at temperatures ranging from 20° C. to 45° C. After swelling for 24 hours at a predetermined temperature, the samples were removed, promptly blotted with filter paper to remove excess surface water, and weighed again (denoted as Ws).
2. The ESR of the hydrogel was calculated using the following formula:

$$ESR = ( Ws - Wd ) / Wd \times 100 \%$$

(Ws represents the mass of the hydrogel at equilibrium swelling, and Wd denotes the mass of the dry gel prior to swelling).
3. The swelling degree of thermoresponsive hydrogels is dependent on the ambient temperature. Upon reaching a specific threshold (the volume phase transition temperature, VPTT), the affinity of the hydrogel for aqueous media decreases abruptly, resulting in rapid liquid expulsion from the pores and marked contraction of the hydrogel. As illustrated in the swelling curve, upon reaching physiological temperature (37° C.), the hydrogel exhibits reduced and stabilized swelling, thereby maintain the sustained release of its encapsulated cytokine or drug payload.

S7. Hydrogel degradation rate assessment, includes the following specific steps.
1. The degradation profile of the hydrogel was assessed gravimetrically. A pre-formed 500 μL thermosensitive HA hydrogel sample was weighed (denoted as Wo) and immersed in 20 mL of PBS (1×PBS, pH 7.4, The system was maintained at 37° C. in a constant-temperature water bath, with the PBS solution replaced daily. At predetermined time intervals, the sample was removed, carefully blotted with filter paper to remove surface moisture, and reweighed (denoted as Wt).
2. The degradation ratio was calculated using the following formula:

$$WR = ( Wo - Wt ) / Wo \times 100 \%$$

(Wo represents the initial mass and Wt denotes the degraded mass).
3. The degradation results show that under physiological temperature (37° C.), the HA hydrogel underwent gradual and sustained degradation over time, exhibiting a relatively constant degradation rate. By day 28, the degradation ratio reached approximately 66.3%. Throughout this process, the encapsulated IFN cytokine was released in a progressive manner.

S8. Synthesis of IFN-loaded sustained-release thermosensitive hydrogel and release kinetics profiling, includes the following specific steps.
1. Synthesis of IFN-loaded thermosensitive hydrogel for sustained release: Based on previous findings from our group regarding the effects of IFN-β₁ and IFN-γ on TM cells, the targeted loading concentrations were set at 2,500-5,000 U/mL for IFN-β₁ and 20-30,000 U/mL for IFN-γ. Each IFN was incorporated into the prepared thermosensitive HA hydrogel solution. The in vitro release profiles were subsequently evaluated under physiologically relevant conditions (37° C.).
2. In vitro release kinetics were assessed as follows: 1 mL IFN-loaded thermosensitive hydrogel were placed in dialysis bags and immersed in 100 mL of PBS solution. The system was maintained at 37° C. under constant agitation at 100 rpm. At predetermined time intervals, 50 μL aliquots of the release medium were collected and replaced with an equal volume of fresh PBS to maintain sink conditions. Following collection, all samples were stored at −20° C. until analysis. The concentrations of IFN-γ and IFN-β₁ were quantified using corresponding ELISA kits. Following collection, samples were stored at −20° C. All ELISA procedures were conducted at 4° C. to prevent IFN degradation and maintain analytical integrity.
3. The release profiles demonstrated that hydrogels loaded with 30,000 U/mL IFN-γ and 5,000 U/mL IFN-β₁ achieved cumulative release rates of 92.0% and 86.7%, respectively, over 28 days. These results confirm the capability of the loaded hydrogel to sustain the release of encapsulated agents for approximately one month or longer, supporting its potential for long-acting release following a single administration.

Example 2, a method for establishing a chronic glaucoma animal model using a thermosensitive hydrogel, including the following steps.

S1. Pre-modeling preparation

S1. Pre-modeling preparation, includes the following specific steps:
1. Experimental animal preparation: C57BL/6J mice (6-8 weeks old, male) were housed in a SPF barrier facility with ad libitum access to food and water. After a 7-day acclimation period, experiments were initiated in strict compliance with institutional animal care and use guidelines. At 08:00-10:00 a.m. on the day prior to surgery, IOP was measured in inhalation-anesthetized mice using a TonoLab rebound tonometer. The mean value was recorded as baseline IOP. All subsequent IOP measurements were performed by the same operator within the identical daily time window.
2. Preparation of IFN-loaded thermosensitive hydrogel for sustained release: The hydrogel was prepared according to the methods described in Steps S1, S2, and S8 of Example 1. The target loading concentrations were 2,500-5,000 U/mL for IFN-β₁ and 20-30,000 U/mL for IFN-γ.
3. Based on the in vitro degradation and release profiles, as well as the animal model establishment method outlined in Step S2 of Example 2, the formulated hydrogels were administered via intracameral injection into the anterior chamber of the mouse eye.

S2. Induction of animal models, includes the following specific steps.
1. Experimental animal groups: The model groups included \textcircled{1} intracameral injection of IFN-γ-loaded sustained-release thermosensitive hydrogel and \textcircled{2} intracameral injection of IFN-β₁-loaded sustained-release thermosensitive hydrogel. The control group received intracameral injection of drug-free thermosensitive hydrogel.
2. Anesthesia of experimental animals: anesthesia was induced in both model and control group mice via intraperitoneal injection of 1% pentobarbital sodium (40-50 mg/kg). The periocular area was disinfected with iodine swabs, eyelashes were trimmed, and the eyeball was exposed. Ocular surfaces were irrigated with sterile PBS or ofloxacin ophthalmic solution, followed by drying with sterile cotton swabs. Prior to surgery, topical 0.5% proparacaine hydrochloride was administered until abolition of withdrawal reflexes confirmed adequate general and local anesthesia.
3. Intracameral injection in experimental animals: a microsurgical technique was employed for intracameral hydrogel delivery. First, the eyeball was stabilized with micro-forceps, a 15° corneal paracentesis knife was used to create a corneal limbal side incision, and aqueous humor was partially drained by the side incision. Then, a 30G needle attached to a micro syringe was inserted through the side incision at a slight angle, and approximately 3 μL of hydrogel was injected into the anterior chamber. This was followed by slow injection of 1 μL air to ensure complete hydrogel delivery and formation of a small intracameral bubble. The needle was maintained in situ for >30 s before gradual withdrawal, allowing the air bubble to self-seal the corneal incision and prevent reflux. The bubble was spontaneously absorbed within hours. Special care was taken to avoid iris or lens capsule injury during the procedure. Postoperatively, ofloxacin ophthalmic ointment was applied to prevent infection. Mice were placed on a thermoregulated recovery pad until spontaneous awakening before returning to housing. Reinjections were performed based on hydrogel degradation kinetics (monitored via slit-lamp) and IOP fluctuations to maintain therapeutic drug concentrations. The total modeling period was 3 months to establish stable chronic ocular hypertension.

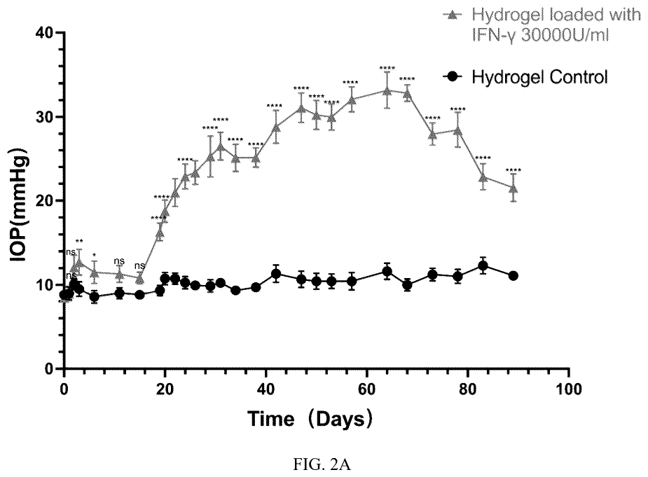

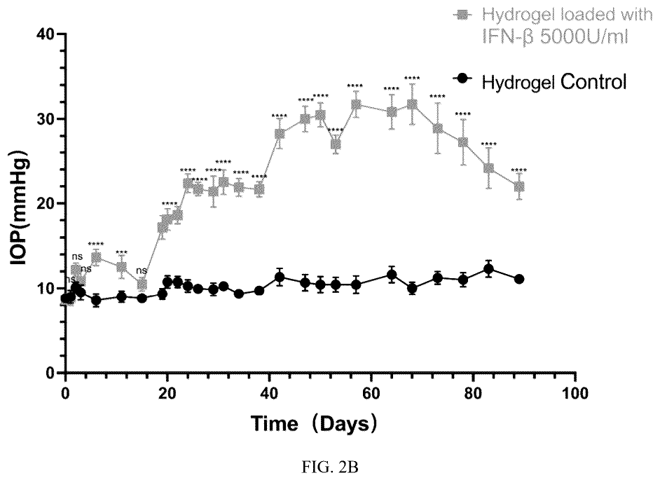

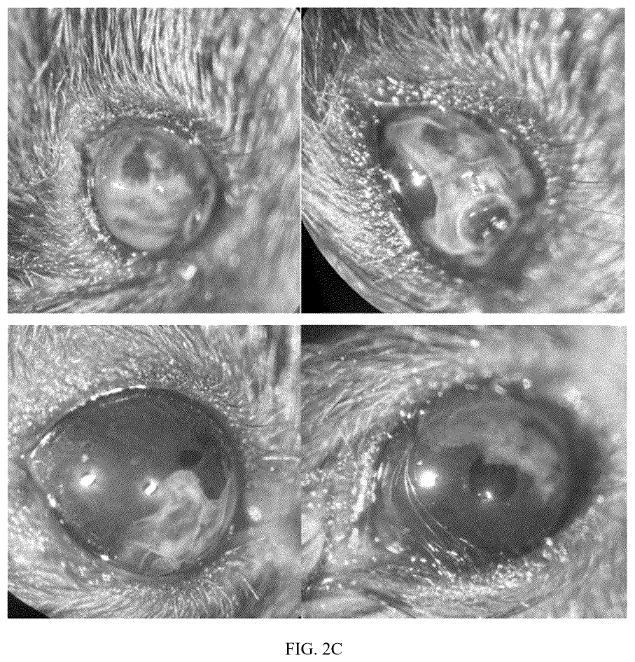

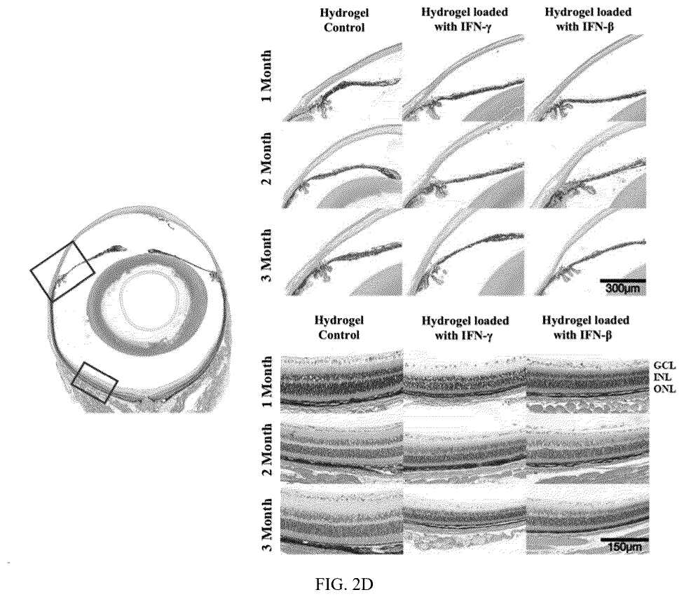

S3. Detection of ocular changes induced in the animal model were consistent with the characteristics of chronic open-angle glaucoma, includes the following specific steps:
1. IOP measurement: IOP was measured in both model and control group mice using a TonoLab rebound tonometer on days 1, 2, 3, 5, 7, 9, 11, 14, 17, 20, 24, and 28 post-modeling. Provided IOP stabilized, measurements were subsequently taken twice weekly during consistent time windows. Based on the degradation profile of the hydrogel in the anterior chamber and observed IOP fluctuations, additional intracameral injections of various IFN-loaded hydrogels were administered to maintain therapeutic IFN concentrations. All acquired IOP data were subjected to statistical analysis to evaluate temporal changes. Results demonstrated a stable and sustained elevation of IOP in the model group following induction, whereas IOP in the control group remained at baseline levels with no significant increase. A statistically significant difference was observed between the two groups, indicating that the present modeling method successfully established chronic ocular hypertension.
2. Distribution of the hydrogel in the anterior chamber was monitored using a ophthalmic surgical microscope immediately after modeling, and at 1, 2, 4, 6, 8, 10, and 12 weeks post-modeling in both model and control group mice. Images were captured for subsequent analysis of gelation status, degradation progression, and any signs of anterior segment inflammation. Results indicated that the hydrogel was initially uniformly distributed in the anterior chamber in both model and control groups (FIG. 2C upper part), followed by gel formation. Residual hydrogel was still observed in the anterior chamber 4 weeks post-modeling (FIG. 2C lower part), demonstrating its gradual degradation over time and the consequent sustained release of the encapsulated drug or cytokine. Furthermore, examination of anterior segment tissues—including the cornea, conjunctiva, iris, anterior chamber, and lens—at all time points revealed no significant inflammatory infiltration.
3. At the end of the first, second, and third months post-modeling, subsets of animals from both the model and control groups were euthanized. The enucleated eyes were processed for paraffin embedding and sectioning. Following hematoxylin and eosin (H&E) staining, the sections were examined and imaged using light microscopy. A comparative histological analysis was conducted to evaluate morphological alterations in the corneal layers, iridocorneal angle configuration, cytoarchitectural organization and cellularity of the retinal layers, depth of the optic disc cupping, and the extent of retrobulbar optic nerve atrophy. Results revealed no significant morphological differences in the corneal layers between the model and control groups, with observable TM structures in the iridocorneal angle, indicating an open-angle configuration. In contrast, the model group exhibited reduced full-thickness retinal thickness, decreased RGCs counts, and in some cases, increased optic disc cupping and retrobulbar optic nerve atrophy. These findings are consistent with characteristic ocular changes associated with OAG.
4. RGCs quantification: At the end of months 1, 2, and 3 post-modeling, subsets of animals from both model and control groups were euthanized via transcardial perfusion. The enucleated eyes were processed for whole-retina flat-mount preparation and immunostaining for RGCs. The posterior pole, mid-periphery, and peripheral regions across all four retinal quadrants were imaged using a ZEISS microscope. Immunopositive RGCs were counted, and the data were subjected to statistical analysis. RGCs loss in the model group, compared to the control group, was assessed to evaluate the extent of optic nerve injury in this ocular hypertension model.
5. TEM of TM structure: At the end of the first, second, and third months post-modeling, subsets of animals from both model and control groups were euthanized. A 2 mm×4 mm tissue segment containing the iridocorneal and TM was dissected from the enucleated eyes. The specimens were fixed in 2.5% glutaraldehyde and processed for TEM to examine the ultrastructural details of the TM. Comparative ultrastructural analysis between the model and control groups suggested that IFN-γ and IFN-β₁ may contribute to the pathogenesis of open-angle glaucoma by potentially inducing TM injury and suppressing cellular activity.
6. Assessment of retrobulbar optic nerve axonal degeneration by immunohistochemistry: At the end of months 1, 2, and 3 post-modeling, subsets of animals from the model and control groups undergo transcardial perfusion. A 1.5-2.0 mm segment of the ON posterior to the globe was dissected and fixed in situ with 2% glutaraldehyde and 2% paraformaldehyde. The specimens were sectioned and stained with 1% PPD. Sections were imaged using a 100× objective on a ZEISS fluorescence microscope to tile the entire ON area without overlap. From the center of each image, at least two non-overlapping regions (21.4 μm×29.1 μm) were selected, and the number of intact axons within these areas was manually quantified. The average number of surviving axons per ON was calculated from all images of a single nerve. The mean number of surviving axons in the model group was compared to that of the control group to determine the percentage of axonal survival. Comparative analysis of axonal structural degeneration between the two groups was also performed.

The present disclosure further provides an innovative intracameral delivery approach utilizing a thermosensitive hyaluronic acid hydrogel loaded with IFN-γ or IFN-β₁. This system enables sustained release following a single local injection. The IFN-loaded hydrogel rapidly gels at physiological temperature (37° C.), exhibits stable swelling, degrades gradually, and releases the IFN progressively over a period of at least 28 days.

Mechanistically, IFN-γ suppresses TM cell viability by regulating necroptosis, whereas IFN-β₁ acts via autophagy activation. Neither cytokine induces anatomical obstruction of the aqueous humor outflow pathway. The gelled hydrogel does not physically block the iridocorneal angle. Upon intracameral injection, the hydrogel enables sustained release of both cytokines, minimizing repeated interventions and reducing potential trauma to anterior segment tissues such as the cornea, iris, and lens.

This model recapitulates key features of chronic OAG, including a significant and sustained elevation in IOP, open iridocorneal angle architecture, and progressive glaucomatous optic neuropathy, thereby providing a novel animal model for the disease.

The present embodiment is merely illustrative of the disclosure and is not intended to limit its scope. Practitioners in the field may make non-inventive modifications to this embodiment based on need after reading this specification, provided that all such adaptations fall within the scope of the claims and are protected under patent law.

What is claimed is:

1. Methods of fabricating a thermosensitive hydrogel, comprising the steps of:
S1, synthesis, dialysis, lyophilization of hyaluronic acid-methacrylic anhydride (HA-MA);
1, synthesis of HA-MA, dissolving 2 g of hyaluronic acid (HA) in 200 mL deionized water, immediately adding 15 mL methacrylic anhydride (MA), adding 5M NaOH, adjusting the pH to between 8-12, and reacting at 2-6° C. for 12 h;
2, dialyzing the synthesized HA-MA conjugate with cellulose ester dialysis membranes for 4 days, with the dialysate replaced three times daily; and
3, pre-freezing the modified HA-MA conjugate to −80° C., and subsequently lyophilizing the pre-frozen modified HA-MA conjugate using a freeze-dryer; and
S2, synthesis, dialysis and lyophilization of thermosensitive hydrogel;
1, weighing 0.2 g of lyophilized HA-MA in step S1 and homogenizing the same with 180 mL of deionized water in a 250 mL round-bottom flask until complete dissolution is achieved;
2, adding 7 mL of 10% (w/v) ethyl methacrylate (EMA) and 7 mL of 10% (w/v) potassium persulfate (KPS) into a round-bottom flask as an initiator system, adding 7 mL of 4% (v/v) N,N,N′,N′-tetramethylethylenediamine (TEMED) pre-deoxygenated with nitrogen as a cocatalyst, into a reaction flask under continuous positive nitrogen pressure, and proceeding at 20° C. for 24 hours to form a copolymer network through copolymerization;
3, dialyzing the synthesized sample with cellulose ester dialysis membranes for 4 days, with the dialysate replaced three times daily; and
4, pre-freezing the synthesized thermosensitive conjugate at −80° C. and subsequently lyophilizing the pre-frozen synthesized thermosensitive conjugate using a freeze-dryer.

2. Induction of a chronic glaucoma animal model via IFN-loaded thermosensitive hydrogel, comprising the steps of:
S1, pre-modeling preparation; and
S2, induction of animal models;
wherein S1 has the specific steps of:
1, preparation of experimental animals: housing C57BL/6J mice (6-8 weeks old, male) in a specific pathogen-free (SPF) barrier facility with ad libitum access to food and water, and after a 7-day acclimation period, initiating experiments in strict compliance with institutional animal care and use guidelines; and at 08:00-10:00 a.m. on the day prior to surgery, measuring intraocular pressure (IOP) in inhalation-anesthetized mice using a TonoLab rebound tonometer, recording a mean value as a baseline IOP, and performing all subsequent IOP measurements by the same operator within an identical daily time window; and
2, preparation of drug-loaded thermosensitive hydrogel for sustained release: target loading concentrations being 2,500-5,000 unit/ml (U/mL) for interferon-β1 (IFN-β₁, and 20-30,000 U/mL for interferon-T (IFN-T); and
S2 has the specific steps of:
1, experimental animal groups: model groups comprising two groups: intracameral injection of IFN-γ-loaded sustained-release thermosensitive hydrogel and intracameral injection of IFN-β₁-loaded sustained-release thermosensitive hydrogel, ad the control group being intracameral injection of drug-free thermosensitive hydrogel;
2, anesthesia of experimental animals: inducing anesthesia in the model groups and the control group mice via intraperitoneal injection of 1% pentobarbital sodium (40-50 mg/kg), disinfecting the periocular area with iodine swabs, trimming eyelashes, and exposing eyeballs; irrigating ocular surfaces with sterile phosphate buffered saline (PBS) or ofloxacin ophthalmic solution, followed by drying with sterile cotton swabs; and prior to surgery, performing topical anesthesia of eyes, administering with 0.5% proparacaine hydrochloride until abolition of withdrawal reflexes, and confirming adequate general and local anesthesia; and
3, intracameral injection in experimental animals: employing a microsurgical technique for intracameral hydrogel delivery, firstly, stabilizing the eyeballs with micro-forceps, creating a corneal limbal side incision using a 15° corneal paracentesis knife, and partially draining aqueous humor through the side incision; inserting a 30G needle attached to a micro syringe through the side incision at a slight angle, and injecting approximately 3 μL of hydrogel into the anterior chamber, followed by slowly injecting 1 μL of air to ensure complete hydrogel delivery and formation of a small intracameral bubble; maintaining the needle in situ for >30 s before gradual withdrawal, allowing the air bubble to self-seal the corneal incision and prevent reflux, the bubble being spontaneously absorbed within hours, and exercising special care to avoid iris or lens capsule injury during the procedure; applying ofloxacin ophthalmic ointment postoperatively to prevent infection, and placing mice on a thermoregulated recovery pad until spontaneous awakening before returning the mice to housing; and performing reinjections based on hydrogel degradation kinetics (monitored via slit-lamp) and intraocular pressure (IOP) fluctuations to maintain therapeutic drug concentrations, and setting a total modeling period to 3 months to establish stable chronic ocular hypertension.
